(19) United States
(12) Patent Application Publication
(10) Pub. No.: US 2025/0049705 A1
(43) Pub. Date: Feb. 13, 2025

(54) PHARMACEUTICAL COMPOSITION COMPRISING TIGECYCLINE

(71) Applicant: ZAMBON S.p.A., Bresso (MI) (IT)

(72) Inventors: Loretta MAGGI, Piacenza (IT); Cristina VENEZIANI, Castel San Giovanni (PV) (IT); Heike BUTTI, Piacenza (IT); Laura ZANELLOTTI, Piacenza (IT); Giovanni TANGHERLINI, Cinisello Balsamo (MI) (IT); Giovanni CAPONETTI, Piacenza (IT)

(73) Assignee: ZAMBON S.p.A., Bresso (MI) (IT)

(21) Appl. No.: 18/719,117

(22) PCT Filed: Dec. 12, 2022
(86) PCT No.: PCT/EP2022/085366

§ 371 Date: Jun. 12, 2024

(30) Foreign Application Priority Data
Dec. 13, 2021 (IT) 102021000031133

(51) Int. Cl. A61K 9/00; A61K 9/16; A61K 31/65; A61P 31/04

(52) U.S. Cl. CPC A61K 9/0075; A61K 9/1623; A61K 31/65; A61P 31/04

(57) ABSTRACT

The present invention refers to a pharmaceutical composition in the form of a dry powder for inhalation administration comprising a glycylcycline, in particular tigecycline, in an amount of less than 50% by weight relative to the total weight of the composition, lactose in an amount equal to or greater than the amount of said tigecycline, and optionally leucine.

### FIELD OF THE INVENTION

The present invention relates to a pharmaceutical composition in the form of a dry powder for inhalation administration provided with high breathability and stability.

In particular, the present invention relates to an inhalation powder indicated for the treatment of pulmonary infections caused by mycobacteria, in particular non-tuberculous mycobacteria, containing drugs belonging to the glycylcyclines class, in particular tigecycline.

### Background Art

Mycobacteria belong to the genus Mycobacterium first identified in 1896. The genus Mycobacterium comprises about 190 bacterial species characterized by a waxy and mycolic acid-rich cell wall that confers resistance to osmotic pressure, to environmental factors and to antibiotics.

With regard to the mycobacterial pulmonary infections, two main etiological agents can be distinguished, the Mycobacterium tuberculosis and the non-tuberculous mycobacteria (NTM, Non Tuberculous Mycobacteria).

The Mycobacterium tuberculosis, the etiological agent of tuberculosis (TB), neatly differs in habitat, pathogenicity, contagiousness and sensitivity to chemotherapeutic agents from all the other species of mycobacteria and remains one of the most common causes of infection and mortality in the world.

Differently, the NTM pulmonary infections (NTM pulmonary disease, NTM-PD) are less known, even if they are slowly becoming one of the main concerns for the global health due to their constant growth in the whole world.

The classification of non-tuberculous mycobacteria has followed two steps. The first step has brought to the classification by using the growth speed as distinctive criterium, by subdividing the mycobacteria in two groups. Colonies observable to the naked eye after 7 days from the seeding were considered belonging to slowly-growing NTM (slowly-growing mycobacteria, SGM), whereas visible colonies in less than 7 days were attributed to rapidly-growing NTM (rapidly-growing mycobacteria, RGM).

The second step of the classification of non-tuberculous mycobacteria has begun in the '90s with the studies of the sequence of the gene codifying for the ribosomal DNA 16S, which have brought to a significant increase of the species number, nowadays nearly 150.

NTM are opportunist pathogens, which mainly cause pulmonary diseases similar to the tuberculosis mostly in immune-compromised patients or patients with pre-existing pulmonary conditions, such as cystic fibrosis (CF), bronchiectasis or chronic obstructive pulmonary disease (COPD).

Although the incidence of tuberculosis started to decrease during the last years (2.3% reduction since 2016), the global prevalence of NTM pulmonary infections has rapidly increased. The yearly one varies in the different areas, and is generally between 0.2/100 000 and 9.8/100 000 with an alarming global growth rate. The situation is worst among vulnerable populations and the growing awareness that the prevalence of NTM in the whole world could be also greater than what is estimated due to wrong diagnoses is cause of further concern.

In developing countries, the wrong diagnosis of NTM mistaken for TB is common, due to their similar aspect at the microscopic examination of sputum smears. This is problematic in many ways: the incidence of NTM is widely underestimated, unnecessarily drains resources dedicated to the global fight against tuberculosis and brings to wrong treatments of patients because the NTM infections do not respond to the classical drug regimens for the tuberculosis.

The mycobacterial pulmonary infections are generally caused by the aerosol inhalation. The source of these aerosols can be environmental, as often occurs for the NTM, or from other infected individuals, as it can be noted for the tuberculosis. Once in the lungs, it is considered that the physiopathology of these infections is relatively similar, although the clinical severity seems to vary. In both cases, the pathogenic invader is rapidly recognized and phagocytized by alveolar macrophages, where mycobacteria survive and proliferate at intracell level. In reply, the human organism recruits its immune system in the form of circulating monocytes, neutrophils, T-cells and dendritic cells to form a granuloma, one of the distinctive marks of mycobacterial pulmonary infections. This strategy often allows the survival of pathogens within the quarantined area leading to tissue cavitation, dissemination and decline of the respiratory function. Therefore, any mycobacterial therapy must be capable of penetrating in this inflammatory environment for effectively targeting the invading pathogenic invaders.

Among NTM that cause pulmonary infections, the Mycobacterium abscessus complex (MABc) is one of the most significant ones associated with pulmonary infections, in particular in patients with cystic fibrosis. The Mycobacterium abscessus complex, first isolated in 1992, has been divided into 3 phylogenetically very similar subspecies: M. abscessus sensu stricto (M. abscessus s.s.), M. massiliensee, and M. bolletii.

The MABc is considered the most pathogenic of the rapidly-growing mycobacteria (RGM). In particular, the MABc is associated with intrinsic and acquired resistance to most anti-mycobacterial agents, including macrolides.

Over the last years, cases of MABc human infection have progressively increased arousing considerable concern in the clinical field. The number of literary works regarding the clinical isolation of MABc from patients with cystic fibrosis, patients with chronic respiratory diseases and patients with bronchiectasis has constantly increased, covering an ever-greater clinical importance. Nowadays, it is considered that MABc is the cause of about 80% of RGM pulmonary infections.

Known treatments for mycobacterial infections are often long and require strict adherence, mainly due to the tenacious nature of mycobacteria and to the development of granulomatous structures. Current therapies for tuberculosis involve the administration of isoniazid, rifampicin, ethambutol and pyrazinamide for 6-30 months. The treatment of NTM pulmonary infections is largely empirical, based on the use of three or four antibiotics, for at least 12 months.

Generally there are prescribed multi-drug regimens based on macrolides (clarithromycin or azithromycin) in combination with parenteral antibiotics, such as for example, an aminoglycoside (streptomycin, neomycin, kanamycin, amikacin and tobramycin), cefoxitin, imipenem or tigecycline, for treatments of at least 12 months, which are often prolonged for 18-24 months.

The treatment of NTM pulmonary infections often involves a significant economic and psychological burden for patients that has as consequence a high rate of treatment interruption. Main causes of treatment interruption are the long duration of the treatment, the lack of observed improvements and the serious side effects associated with oral and parenteral administration.

For the above-mentioned reasons, there is an urgent medical need to develop more effective and safer regimens constituted by more bioavailable drugs for the treatment of NTM pulmonary infections, which led to the development of treatments with antibiotics by inhalation.

Nowadays, the administration of drugs by inhalation is obtained through delivery with inhaler devices such as:
nebulizers, for which the drug is dissolved or dispersed in suspension form, and conveyed in the lung as fine nebulized droplets;
pressurized inhalers, through which the drug-still in the form of solution or suspension droplets-is conveyed in the lung by an inert gas rapidly expanded in air from a pressurized can;
powder inhalers, capable of delivering the drug in the inhaler and conveying it into the lung as micronized dry particles.

In the international patent application published with number WO2020/239696 it has been suggested the use of a glycylcycline, in particular tigecycline, in the treatment of mycobacterial infections via inhalation. WO2020/239696 describes the use of solutions for nebulizers and powders for inhalers, the latter realized according to the process described in WO2011/073002 and comprising 98-99.9% of glycylcycline and 0.1-2% of a lubricant (in particular magnesium stearate).

There are also known solutions of a glycylcycline, in particular tigecycline, in the treatment of mycobacterial infections by parenteral route, as described in WO2006/099258, obtained by reconstitution in aqueous solution of a powder comprising tigecycline, a carbohydrate selected from lactose, mannose, sucrose and glucose, and an acid and/or a buffer in an amount to provide the solution with a pH between 4 and 5. WO2014/032956 describes an example of preparation of a tigecycline-lactose powder by the in-vial freeze-drying technique of a solution of tigecycline and lactose with pH between 7.2 and 7.7.

Nevertheless, glycylcyclines, and in particular tigecycline, are active ingredients that present formulation technical difficulties such that, nowadays, despite the fact that these active ingredients have been known for over forty years, there are only formulations in solution for intravenous infusion in a hospital setting, and there are no formulations, either under approval or approved by regulatory authorities, suitable for administration by inhalation.

The in-vial freeze-drying techniques used to make tigecycline powders for reconstitution in aqueous solution for parenteral administration, such as those described in WO2006/099258 and WO2014/032956, do not allow to obtain the size and aerodynamic properties required for inhalation administration in powder form. A powder obtained by freeze-drying is not suitable for a direct administration via inhalation.

The inhalation formulations in the form of powder have conventionally been obtained through the grinding/micronization of active ingredients in crystalline form in order to obtain particles generally less than 5.0 μm in diameter, more preferably less than 2.0 μm. In general, the use of excipients has been limited to solving problems of powder flowability of micronized active ingredients.

It is evident that the formulation technique based on grinding/micronization has several limitations from the standpoint of being able to process active ingredients, including those that differ widely in chemical and physicochemical characteristics, while ensuring that the final formulation possesses aerodynamic properties suitable for inhalation delivery to the deep regions of the respiratory system.

In this regard, an effective approach for obtaining inhalant powders with good aerodynamic properties is represented by particle engineering, achievable through the production technique of spray drying. According to this technique, the active ingredient and suitable excipients can be combined to form particles whose aerodynamic properties are defined by the composition and by the process conditions adopted.

Despite the opportunities offered by particle engineering, this technique is not without formulation difficulties to overcome. Among the most significant ones encountered in the development of powder inhalation products is certainly the need to ensure sufficient chemical stability for the product under development during the execution of the production process.

The stability of an inhalation product is particularly important in relation to the fact that it must be administered into the deep lung while retaining its physical characteristics for a quantitative penetration of particles down to the deepest regions of the lung. To this must be added the fact that the number of excipients currently approved for inhalation administration, and therefore acceptable in terms of toxicity towards lung tissue, is extremely limited.

### SUMMARY OF THE INVENTION

In view of all the above considerations, it would be advantageous to make a pharmaceutical composition for inhalation administration in dry powder form comprising a glycylcycline, in particular tigecycline, that is stable and easily administrable with common dry powder inhalers, while maintaining ease of implementation.

In the art, the problem of providing an inhalation formulation comprising a glycylcycline, in particular tigecycline, that is stable and can be administered with common dry powder inhalers, while retaining characteristics of high deliverability and breathability, and that can be produced industrially in an economically advantageous process, remains in fact unsolved or unsatisfactorily solved.

The Applicant therefore has addressed the technical problem of making an inhalation formulation comprising a glycylcycline, in particular tigecycline, for the treatment of non-tuberculous mycobacteria pulmonary infections, with particular interest in infections caused by mycobacteria belonging to the species Mycobacterium Abscessus Complex.

In particular the Applicant has addressed the problem of obtaining a high stability of the tigecycline, both during the production process and in the finished product in the form of a dry powder.

At the same time, the Applicant has addressed the problem of ensuring a high breathability in order to reach, in high therapeutic amounts, the deeper regions of the lung, identified as the most distal bronchial and alveolar region with the purpose of reaching the alveolar macrophages and being able to penetrate within them through a mechanism of direct and rapid permeation through the cell wall.

The Applicant has observed that the use of lactose in a solution containing tigecycline maintained at pH 7 through the addition of an acidic compound, in particular an organic or inorganic acid, preferably volatile at the working temperatures of the drying process, made it possible to obtain a stable solution during the powder production process by means of drying and at the same time realized a dry powder that was stable over time and with optimal breathability characteristics.

In particular, the Applicant has observed that these results were obtained when the amount of lactose was equal to or greater than the amount of tigecycline.

The Applicant has also observed that the addition of leucine to the solution used in the drying process further improved the breathability and flowability characteristics of the resulting dry powder as well as its stability against environmental humidity.

Therefore, in a first aspect, the present invention relates to a pharmaceutical composition in the form of a dry powder for inhalation administration comprising a glycylcycline, in particular tigecycline, in an amount of less than 50% by weight relative to the total weight of the composition, and lactose in an amount equal to or greater than the amount of said glycylcycline.

Advantageously, the pharmaceutical composition according to the first aspect of the present invention also comprises leucine.

The Applicant has observed that the tigecycline present in the dry powder according to the present invention was substantially in an amorphous form.

In a second aspect, the present invention also relates to a process for preparing a pharmaceutical composition in the form of a dry powder for inhalation administration comprising a glycylcycline, in particular tigecycline, wherein said process comprises the following steps:
(a) preparing a solution in an aqueous solvent comprising a glycylcycline, in particular tigecycline, lactose, and optionally leucine;
(b) drying the solution of step (a) to obtain a dry powder with X₉₀ lower than 10 μm, and
(c) collecting said dry powder,
characterized in that said solution further comprises an organic or inorganic acid in an amount necessary to give said solution a pH value between 6.5 and 7.5, preferably about 7.0.

In a third aspect, the present invention relates to a pharmaceutical composition in the form of a dry powder for inhalation administration for use in the treatment of mycobacterial infections, in particular non-tuberculous mycobacteria, wherein said composition comprises a glycylcycline, in particular tigecycline, in an amount of less than 50% by weight relative to the total weight of the composition, and lactose in an amount equal to or greater than the amount of said glycylcycline.

In a fourth aspect, the present invention relates to a method for the treatment of mycobacterial infections, in particular non-tuberculous mycobacteria, in a subject in need thereof that comprises the administration by inhalation of an effective amount of a pharmaceutical composition in the form of a dry powder that comprises a glycylcycline, in particular tigecycline, in an amount of less than 50% by weight relative to the total weight of the composition, and lactose in an amount equal to or greater than the amount of said glycylcycline.

### BRIEF DESCRIPTION OF THE FIGURES

The description will be now hereinafter indicated with reference to the attached figures, provided for indicative purposes only and, therefore, not limiting, wherein:

FIG. 1 shows the formation of degradation products of an increasingly intense color after 24 hours of storage of a solution of tigecycline at 5° C., at 25° C. and at 40° C. respectively;

FIG. 2 shows the diffractogram obtained with a powder obtained by spray drying, comprising tigecycline and lactose in a 1:1 ratio (curve A) and with a powder comprising tigecycline, lactose and leucine in a 1:1:1 ratio (curve B):

FIG. 3 shows an electron microscope photo of a sample of formulation 1 (3A) and of formulation 2 (3B) described in the example 1.

### DETAILED DESCRIPTION OF THE INVENTION

The present invention relates to a pharmaceutical composition in the form of a dry powder for inhalation administration comprising a glycylcycline, in particular tigecycline, in an amount of less than 50% by weight relative to the total weight of the composition, and lactose in an amount equal to or greater than the amount of said glycylcycline.

According to the present invention with the expression “powder for inhalation administration” is meant a powder suitable for pulmonary administration. A powder for inhalation administration can be dispersed and inhaled by means of a suitable inhaler, so that the particles of which it is composed can penetrate within the lungs up to the alveoli in order to carry out the pharmacological characteristics of the active ingredient of which it is composed. Particles with an aerodynamic diameter of less than 5.0 μm are considered to be normally inhalable.

According to the present invention with the expression “dry powder” is meant a powder that has a humidity content of less than 10%, preferably less than 5%, more preferably less than 3%.

Advantageously, the glycylcycline used in the pharmaceutical composition of the present invention is the tigecycline. Other glycylcyclines useful in the pharmaceutical composition of the present invention are the eravacycline, and other experimental glycylcyclines known with the acronym DMG-DMDOT, DMG-MINO, and DMG-DOXY.

The amount of tigecycline present in the pharmaceutical composition in the form of a dry powder of the present invention is preferably from 15% to 45%, more preferably from 20% to 40%, even more preferably from 25% to 35% by weight relative to the total weight of the composition.

The amount of lactose present in the pharmaceutical composition of the present invention is preferably of 30% to 85%, more preferably of 35% to 80%, even more preferably of 40% to 75% by weight relative to the total weight of the composition.

Advantageously, the weight ratio between the amount of tigecycline and the amount of lactose present in the pharmaceutical composition according to the present invention is between 1:1 and 1:3, preferably between 1:1 and 1:2.

Advantageously, the pharmaceutical composition of the present invention also comprises leucine.

According to a preferred aspect of the present invention, the pharmaceutical composition of the present invention comprises leucine in an amount of 5% to 30%, preferably of 10% to 25% by weight relative to the total weight of the composition.

Leucine is a natural amino acid whose local tolerability characteristics following inhalation are now widely recognized and documented, even though an inhalation powder containing leucine as an excipient has not yet been introduced onto the market.

Leucine is a hydrophobic amino acid, and the Applicant has observed that during the drying process, leucine tends to deposit on the surface of the particles forming a substantially hydrophobic layer that enhances the humidity resistance and the flowability of the produced particles.

Advantageously, the pharmaceutical composition of the present invention comprises tigecycline in amounts between 20% and 40%, preferably between 25% and 35%, lactose in amounts between 30% and 80%, preferably between 40% and 70% by weight relative to the total weight of the composition, and leucine for the remaining part in the amount necessary to reach the 100% by weight.

In a particularly preferred aspect, the pharmaceutical composition of the present invention comprises tigecycline in amounts of about 30%, lactose in quantities between 45% and 60%, and leucine in quantities between 25% and 10% by weight relative to the total weight of the composition.

The pharmaceutical composition according to the present invention has preferably a size distribution (X₉₀) so that at least 90% of the particles has an equivalent diameter of less than 10.0 μm, preferably less than 7.0 μm, more preferably less than 5.0 μm.

The Applicant has observed that the lower is the value of X₉₀, the greater is the surface area of the powder, and the deeper is the pulmonary deposition.

In particular, the pharmaceutical composition in powder according to the present invention has a median aerodynamic diameter of particles (Mass Median Aerodynamic Diameter—MMAD) lower than 5 μm, preferably of 2 μm to 4 μm.

Advantageously, the pharmaceutical composition in powder according to the present invention has a respirable fine particle fraction (FPF) greater than 50%, preferably greater than 60%, more preferably greater than 70%.

With the term of “fine particle fraction (FPF)” is meant the fraction of powder, relative to the total powder delivered by an inhaler, that has an aerodynamic diameter (dae) of less than 5.0 μm. With the term of “delivered fraction (DF)” is meant the fraction of delivered active ingredient, relative to the total charged one. The characterization test that is performed to assess these powder properties is the Next Generation Impactor (NGI) as described in the current ed. of European Pharmacopoeia. According to the present invention, the conditions for executing this test consist of subjecting the powder to an aspiration through the inhaler such that a flow of 60±2 liters/min is generated. This flow in the case of the inhaler mod. RS01 (Plastiape, Osnago IT) is obtained by generating a pressure drop in the system of 1.4 KPa.

According to a preferred aspect, the tigecycline and the lactose present in the pharmaceutical composition in powder according to the present invention are substantially in amorphous form, while leucine, when present, is substantially in crystalline form.

According to the present invention with the expression “substantially in amorphous form” is meant that the percentage of tigecycline or lactose in amorphous form is between 51-100%, preferably between 70-100% and even more preferably between 90-100% relative to the total amount of tigecycline or lactose in the pharmaceutical composition in powder.

According to the present invention with the expression “substantially in crystalline form” is meant that the percentage of leucine in crystalline form is between 51-100%, preferably between 70-100%, more preferably 80-100%, and even more preferably between 90-100% relative to the total amount of leucine in the pharmaceutical composition in powder.

According to the present invention the powder can also comprise excipients suitable for inhalation administration.

These excipients are preferably surfactants, such as for example, non-ionic surfactants such as polysorbates and polyoxyethylene and polyoxypropylene block copolymers, known as “Poloxamers”, in particular the polysorbate 80 known as “Tween 80”, sugars such as for example lactose, mannitol, sucrose, trehalose, maltodextrins and cyclodextrins; fatty acids; fatty acid esters; lipids, preferably phospholipids such as, for example, natural and synthetic sphingophospholipids as well as natural and synthetic glycerophospholipids including diacyl-phospholipids, alkyl-acyl phospholipids and alkenyl-acyl phospholipids; amino acids; and peptides such as dileucine and trileucine or hydrophobic proteins.

The presence of a surfactant is useful to ensure the removal of electrostatic charges eventually present in the formulations without it, the presence of fatty acids and other lipidic substances is useful for ensuring the smoothness of the powder, and the presence of additional sugars can be useful for further powder coating.

Advantageously, excipients capable of reducing residual humidity of the powder, such as for example excipients of hydrophobic nature, are particularly useful for improving the stability of the pharmaceutical composition of the present invention.

In a second aspect, the present invention relates to a process for preparing a pharmaceutical composition in the form of a dry powder for inhalation administration comprising a glycylcycline, in particular tigecycline, wherein said process comprises the following steps:
(a) preparing a solution in an aqueous solvent comprising a glycylcycline, in particular tigecycline, lactose, and optionally leucine,
(b) drying the solution of step (a) to obtain a dry powder with X₉₀ lower than 10 μm, and
(c) collecting said dry powder,
characterized in that said solution further comprises an organic or inorganic acid in an amount necessary to give said solution a pH value between 6.5 and 7.5, preferably about 7.0.

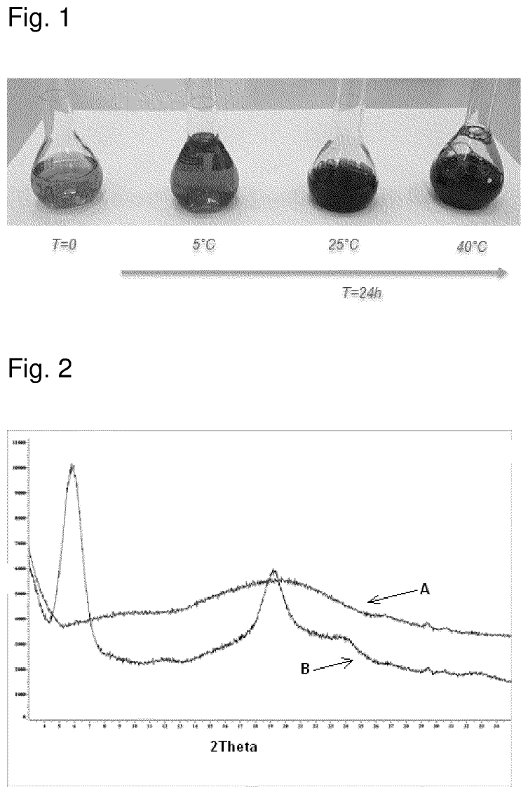

Tigecycline is an extremely unstable active ingredient in aqueous solution wherein it undergoes degradation phenomena mainly by oxidation and epimerization as shown in FIG. 1, which shows the formation of degradation products of increasingly intense color after 24 hours of storage of a solution of tigecycline at 5° C., at 25° C. and at 40° C. respectively.

The Applicant has observed that an aqueous solution comprising tigecycline, lactose, and optionally leucine within which an organic or inorganic acid is added in sufficient amount to give said solution a pH value between 6.5 and 7.5, preferably about 7.0, permits to carry out the drying process without any degradation phenomena of tigecycline.

Preferably, organic or inorganic acid is a volatile compound at the working temperatures of the drying process, and in particular is selected from the group that comprises formic acid, acetic acid, propionic acid, butyric acid, hydrochloric acid, bromic acid, nitric acid and phosphoric acid.

Formic acid is particularly preferred because the Applicant has observed that at the working temperatures of the drying process, formic acid decomposes into carbon dioxide and water, leaving no trace in the resulting powder.

Advantageously, step (a) of preparing the solution is performed away from light and at a temperature equal to or less than 20° C., preferably less than 10° C., and more preferably between 0° and 5° C.

Preferably, the solvent used is constituted by water, advantageously demineralized water, distilled water, sterile water, or deionized water, but water-alcohol mixtures with a water: alcohol ratio between 70:30 v/v and 30:70 v/v can also be used.

Advantageously, the solvent used is opportunely degassed so as to have a dissolved oxygen content of less than 10%, preferably less than 5%, more preferably less than 3%.

The Applicant has observed that the decrease in oxygen content in the solvent permits to reduce the oxidation phenomena of tigecycline, resulting in greater stability of it in the solution prepared for the drying process.

The preferably used alcohols are selected from the group that comprises methanol, ethanol, 1-propanol, 2-propanol, 2-methyl-1-propanol, 1-butanol, 2-butanol, 3-methyl-1-butanol, 1-pentanol alone or in a mixture. The use of ethanol is particularly preferred.

Advantageously, the solution is prepared by adding a glycylcycline, in particular tigecycline, lactose, and optionally leucine to the solvent used, preferably water, and then by adjusting the pH to the desired value, preferably to about 7.0, through the addition of an aqueous solution at 10% of formic acid.

According to a particularly preferred embodiment of the second aspect of the present invention, the step (a) comprises the following steps:
(a1) degassing said aqueous solvent until a dissolved oxygen content of less than 10% by weight is obtained;
(a2) adding to said aqueous solvent said lactose, and optionally said leucine, and after complete dissolution of said lactose, and optionally of said leucine, adding said glycylcycline, in particular tigecycline;
(a3) adding an organic or inorganic acid in sufficient amount to give said solution a pH value of about 7.0;
(a4) cooling said solution at a temperature below 5° C. in a container sealed from the light.

Advantageously, step (b) of the preparation process according to the present invention is carried out with the technique of spray drying using a spray dryer.

The Applicant has observed that the spray drying permits to obtain a dried powder with uniform and substantially amorphous particles.

In particular, the Applicant has observed that the spray drying process allows to obtain powders consisting of very fine, inhalable particle size through a drying mechanism of a properly atomized solution in a controlled inlet and outlet temperature environment, which occurs in very few hundredths of a second ensuring a substantial stability of the powder obtained after the process.

The feed rate at which it is possible to operate in order to obtain a dried powder with the desired characteristics according to the invention, is given by the type of spray drying that is used, i.e. an industrial-sized spray dryer or a “pilot”-sized spray dryer, or also a laboratory spray dryer.

Advantageously, the Applicant has observed that in laboratory spray dryers the step (b) of spray drying gives optimal results with a feed rate equal to or greater than 3 g/minute, whereas on pilot-sized systems optimal results are obtained with a feed rate equal to or greater than 10 g/minute, preferably greater than 15 g/minute, even more preferably equal to or greater than 20 g/minute.

Generally, the feed rate used by industrial-sized spray dryers is generally between 150 and 200 g/minute, but there are no limits if spray dryers of greater sizes were used.

Advantageously, the step (b) of spray drying is performed at an inlet temperature between 80° and 200° C., advantageously between 90° and 160° C.

With the term inlet temperature according to the present invention is meant the temperature that the solution meets at the nozzle outlet of the spray dryer, at the inlet of the drying chamber.

Preferably, the step (b) of spray drying is performed at an outlet temperature between 40° and 120° C., advantageously between 50° and 100° C.

With the term outlet temperature according to the present invention, is meant the temperature of the already dried product after it came out from the drying chamber, before entering the cyclone separator.

In a third aspect, the present invention relates to a pharmaceutical composition in the form of a dry powder for inhalation administration for use in the treatment of mycobacterial infections, wherein said composition comprises a glycylcycline, in particular tigecycline, in an amount of less than 50% by weight relative to the total weight of the composition, lactose in an amount equal to or greater than the amount of said tigecycline, and optionally leucine.

In particular, the pharmaceutical composition of the present invention finds application in the treatment of infections caused by Mycobacterium tuberculosis and by non-tuberculous mycobacteria (NTM), preferably selected from the group consisting of slowly-growing NTM (SGM) and rapidly-growing NTM (RGM).

In particular, slowly-growing non-tuberculous mycobacteria (SGM) are selected from the group consisting of Mycobacterium avium complex (MAC), Mycobacterium avium, Mycobacterium intracellulare, Mycobacterium chimaera, Mycobacterium xenopi, Mycobacterium simiae, Mycobacterium marinum and Mycobacterium kansasii.

Advantageously, rapidly-growing non-tuberculous mycobacteria (RGM) are selected from the group consisting of Mycobacterium abscessus, Mycobacterium fortuitum, Mycobacterium abscessus sensu stricto, Mycobacterium massiliensee, Mycobacterium bolletii, Mycobacterium peregrinum, Mycobacterium chelonae and Mycobacterium abscessus complex.

The present invention will be further illustrated hereinafter by means of a certain number of preparatory examples, which are provided for purely indicative purposes and without any limitation of the present invention.

### Example 1

### Preparation of the Solution to be Dried

For the preparation of the pharmaceutical composition in the form of a dry powder of the present invention an aqueous solution comprising tigecycline and excipients indicated in the following tables was used in order to have a solid concentration between 5 and 5.5% by weight, whose pH value was brought to about 7.0 with a 10% formic acid aqueous solution.

The preparation process of the tigecycline aqueous solution comprises various steps and measures, in order to maintain the chemical stability during the whole drying step.

The water used as solvent underwent a degassing process, with the purpose of eliminating the dissolved oxygen, through nitrogen stream at the flow of (12 l/min for 25 minutes in case of 100 ml of solution), until an oxygen content of less than 10% is obtained, measured through a probe (Oxygen meter Hanna HI98198).

The excipients are dissolved in degassed water, and after the complete dissolution, the active ingredient (Tigecycline) is added in the amount indicated in each example, equal to a solid concentration between 5% and 5.5% by weight.

After dissolution of the active ingredient, the solution is adjusted at pH 7.00 by adding formic acid.

The so obtained solution is kept chilled at a temperature equal to or less than 5° C., within an obscured container in order to protect it from the light to which the active ingredient is sensitive.

### Preparation of the Powder Composition

The so obtained solution has been processed by means of a Spray Dryer ProCepT apparatus, by setting the following process parameters:
nozzle of diameter 0.6 mm for the outlet of the solution
atomization gas: air
atomization pressure: 3 bar
drying gas: air
drying gas flow: 0.35 m³/min
inlet temperature: 90° C.
outlet temperature: 45-47° C.
feed rate: 3 g/min
system of powder collection: cyclone separator

At the end of the drying process, immediately after the production, the powder composition was packed under nitrogen atmosphere in glass containers, which in turn were stored in heat-sealed aluminum bags.

The following tables show a series of comparison and invention examples performed according to the above-mentioned specifications.

Table 1 summarizes the results of the analytic determination and of the particle size analysis of the particles obtained with the composition indicated in the first column of table 1.

TABLE 1

| | WC | X₉₀ | TGC | Impurities | FPF |
|---|---|---|---|---|---|
| Composition % | % | μm | Titre % | % | % |
| TGC 100% | 4.8 | 3.3 | 94.7 | 2.8 | 91.4 |
| TGC:LEU 50:50 | 3.3 | 3.5 | 95.4 | 2.3 | 88.4 |
| TGC:LAT 50:50 | 5.4 | 4.8 | 100.6 | 0.9 | 73.6 |
| TGC:ALB 50:50 | 5.8 | 4.2 | 96.9 | 2.2 | 80.1 |
| TGC:LAT:LEU | 4.0 | 4.0 | 99.4 | 1.2 | 75.8 |
| 33:33:33 | | | | | |
| TGC:ALB:LEU | 4.3 | 5.0 | 98.7 | 1.8 | 69.5 |
| 33:33:33 | | | | | |
| TGC:LAT:ALB | 5.3 | 7.0 | 100.9 | 1.0 | 58.6 |
| 33:33:33 | | | | | |

TGC: Tigecycline
LEU: Leucine
LAT: Lactose
ALB: Albumin
WC: Water content
X₉₀: Diameter below which 90% of particles lie
FPF: Fine particle fraction less than 5 μm

The results resumed in table 1 related to the analytic determination of tigecycline and of the impurities present in the resulting powder showed that the presence of lactose is necessary for the stability of tigecycline, while the presence of leucine or albumin alone is not sufficient to stabilize the tigecycline, with consequent formation of impurities.

At the same time, the results resumed in table 1 related to the water content showed that the presence of leucine allows to obtain a drier powder with a lower water content, less than 5% and even equal to or less than 4%.

On the contrary, the results resumed in table 1 showed that the presence of albumin, even in combination with lactose, did not permit to obtain the water content and the X₉₀ value desired, giving the lowest values of FPF %.

Finally, the results of the particle size analysis together with their aerodynamicity (X₉₀ and FPF %) showed that all the combinations, with the exception of the ternary combination TGC: LAT: ALB gave good results.

Table 2 summarizes the results of the analytic determination and of the particle size analysis of the same particles after storage at room temperature (25° C. and 60% RH) for 9 months and at 4° C. for 15 months.

TABLE 2

| | 9 months, room temperature | | | | 15 months, 4° C. | | | |
|---|---|---|---|---|---|---|---|---|
| | WC | X₉₀ | TGC | Impurities | WC | X₉₀ | TGC | Impurities |
| Composition | % | μm | % | % | % | μm | % | % |
| TGC 100% | 6.4 | 3.2 | 81.9 | 18.1 | 5.1 | 3.1 | — | — |
| TGC:LEU 1:1 | 4.3 | 3.8 | 84.4 | 15.6 | 3.9 | 3.5 | — | — |
| TGC:LAT 1:1 | 7.0 | 4.6 | 95.9 | 4.1 | 5.7 | 4.6 | 98.3 | 1.7 |
| TGC:ALB 1:1 | 7.0 | 4.1 | — | — | 6.4 | 4.0 | — | — |
| TGC:LAT:LEU 1:1:1 | 5.3 | 4.1 | 95.9 | 4.1 | 4.3 | 4.0 | 98.2 | 1.8 |
| TGC:ALB:LEU 1:1:1 | 5.4 | 5.3 | — | — | 4.7 | 5.0 | — | — |
| TGC:LAT:ALB 1:1:1 | 6.8 | 6.6 | — | — | 6.0 | 6.3 | — | — |

TGC: Tigecycline
LEU: Leucine
LAT: Lactose
ALB: Albumin

The results of Table 2 confirmed the long-term stability of the compositions comprising lactose, after storage at 4° C. or at room temperature.

Table 3 summarizes the results of the analytic determination of the particles obtained with the composition indicated in the first column of table 1 after in bulk or in capsule storage for 1 or 3 months at various conditions of temperature or relative humidity.

TABLE 3

| | Bulk | | Standard capsules | | Anhydrous capsules | |
|---|---|---|---|---|---|---|
| | 40° C. 75% RH | 30° C. 65% RH | 25° C. 60% RH | | 25° C. 60% RH | |
| Composition | 1 month | 1 month | 1 month | 3 months | 1 month | 3 months |
| TGC:LAT 1:2 | KO | OK | OK | KO | OK | OK |
| TGC:LAT 1:1.5 | KO | OK | OK | KO | OK | OK |
| TGC:LAT 1:1 | KO | OK | OK | KO | OK | OK |
| TGC:LAT 2:1 | KO | KO | KO | — | KO | — |
| TGC:LAT 3:1 | KO | KO | KO | — | KO | — |
| TGC:LAT 4:1 | KO | KO | KO | — | KO | — |

TGC: Tigecycline
LEU: Leucine
LAT: Lactose
RH: Relative humidity
KO: Failed test, formation of impurities greater than 5%
OK: Test successful, stable product

The results of Table 3 highlighted the need of having an amount of lactose equal to or greater than the amount of tigecycline, with a weight ratio between the amount of tigecycline and the amount of lactose ranging from 1:1 to 1:2.

Table 4 summarizes the results of the analytic determination of the particles obtained with the composition indicated in the first column of table 1 after storage in bulk or in dried capsules after filling in nitrogen atmosphere, for 1 month at various conditions of temperature or relative humidity.

TABLE 4

| | | 40° C. 75% RH | | 30° C. 65% RH | |
|---|---|---|---|---|---|
| Composition % | TGC:LAT | Bulk | Capsule | Bulk | Capsule |
| TGC:LAT:LEU | 1:0.8 | KO | OK | OK | OK |
| 50:40:10 | | | | | |
| TGC:LAT:LEU | 1:1 | KO | OK | OK | OK |
| 40:40:20 | | | | | |
| TGC:LAT:LEU | 1:1.5 | KO | OK | OK | OK |
| 30:45:25 | | | | | |
| TGC:LAT:LEU | 1:2 | KO | OK | OK | OK |
| 30:60:10 | | | | | |
| TGC:LAT:LEU | 1:2 | KO | OK | OK | OK |
| 25:50:25 | | | | | |

TGC: Tigecycline
LEU: Leucine
LAT: Lactose
RH: Relative humidity
KO: Failed test, formation of impurities greater than 5%
OK: Test successful, stable product

The results of Table 4 confirmed the usefulness of leucine for the improvement of the long-term stability of the powder, which thereby successfully passed the test at 30° C. and 65% relative humidity and also the most extreme test at 40° C. and 75% relative humidity in dried capsules after filling in nitrogen atmosphere.

Also in this case, the best results are obtained with a tigecycline:lactose ratio between 1:1 and 1:2, and it was observed from the results of the particle size analysis (X₉₀ and FPF %) that the optimal amount of tigecycline resulted to be not greater than 30% by weight.

The following table 5 summarizes the results of the analytic determination and of the particle size analysis of two ternary formulations comprising 30% by weight of tigecycline and optimal amounts of lactose and leucine according to the present invention.

The formulation 1 comprised TGC:LAT:LEU in percentage ratio equal to 30:45:25 with a tigecycline:lactose ratio of 1:1.5 whereas the formulation 2 comprised TGC:LAT:LEU in percentage ratio equal to 30:60:10 with a tigecycline:lactose ratio of 1:2.

TABLE 5

| | | Formulation 1 | Formulation 2 |
|---|---|---|---|
| TGC:LAT:LEU % | | 30:45:25 | 30:60:10 |
| TGC:LAT | | 1:1.5 | 1:2 |
| BULK WC % | | 4.4 | 4.7 |
| CPS WC % | | 2.6 | 2.0 |
| TGC % | | 101.7 | 99.7 |
| Impurities % | | 1.1 | 0.9 |
| PSD | X₅₀ | 2.5 | 2.2 |
| | X₉₀ | 5.0 | 4.3 |
| | VMD | 2.7 | 2.4 |
| PA | FPF | 62.1 | 73.7 |
| | MMAD | 3.7 | 2.6 |
| | GSD | 1.77 | 1.83 |

TGC: Tigecycline
LEU: Leucine
LAT: Lactose
BULK WC %: Water content in bulk
CPS WC %: Water content in capsules
PSD: Particle size distribution
VMD: Volume mean diameter
PA: Aerodynamic parameters
FPF: Fine particle fraction
MMAD: Mass Median Aerodynamic Diameter
GSD: Geometric standard deviation

The data of table 5 confirmed the optimal results obtained with both the compositions 1 and 2, both from the point of view of breathability, with optimal values of PSD and PA, and from the point of view of stability, with optimal values of tigecycline and minimal amounts of impurities.

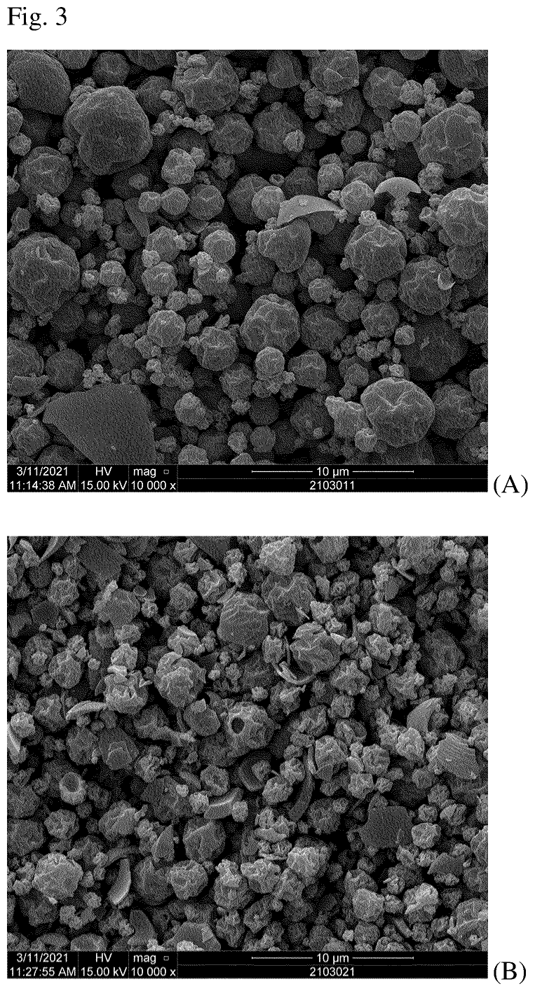

FIG. 3 shows an electron microscope photo of a sample of the formulation 1 (3A) and of the formulation 2 (3B).

### Example 2

### Description of Used Analytic Methods

The values indicated in the preceding tables were determined with the following methods.

### Characterization of the Powder Composition

### 1. Particle Size Analysis

Powder compositions obtained were characterized in terms of dry particle size using a Laser Diffraction Sympatec HELOS/BR apparatus, capable of analyzing the sizes of particles, equipped with the RODOS/L dispersion system for the analysis of powders, associated to the ASPIROS/L system for the automatic loading of the sample.

The instrument was calibrated with reference material and prepared following the instructions given in the user manual of the tool.

### Analysis Process:

The product was sampled in opportune sample-holder (vial) through Aspiros and analyzed.

The dispersion gas used was compressed air opportunely freed from particles.

The method with which the Particle Size Distribution analysis was performed was the following:
Analysis tool: Laser Light Diffraction Particle Sizer Sympatec HELOS/BR
Lens: R1 (0.1-35 μm)
Sample dispersion system: RODOS/L
Sample feed system: ASPIROS/L
Dispersion pressure: 3 bar, with depressurization auto-adjustment
Signal integration time: 10.0 s
Duration of the reference measurement: 10.0 s
Measurement valid in the concentration range of channel 20 from 1.5% to 50%
Software version: PAQXSOS 3.1.1
Calculation method: FREE

All the analyses were performed in an environment with room temperature and humidity.

The particle size analysis returns the diameter values below which the 50% of population (X₅₀), the 90% of population (X₉₀) and the volume mean diameter (VMD) of the particle population in the powder composition sample respectively fall.

### 2. Determination of the Active Ingredient and of the Other Components in the Powder Composition

For the determination of the content of active ingredient and of the other components in the powder composition, an HPLC (High Performance Liquid Chromatography) analytic method was used.

The analytic method used is characterized by the following parameters:
Solvent: 80/20 phosphate buffer pH 8/acetonitrile
Mobile phase: acetonitrile/phosphate buffer pH 6.4

Gradient elution

| | | % buffer |
|---|---|---|
| Time (min) | % Acetonitrile | pH 6.4 |
| 0 | 11.8 | 88.2 |
| 2 | 11.8 | 88.2 |
| 42 | 24.4 | 75.6 |
| 57 | 50 | 50 |
| 60 | 50 | 50 |

+10 min post time

Flow: 1 ml/min
Injection volume: 25 μl
Analysis column: Agilent Pursuit XRs C18, 150 mm×4.6 mm, 3 μm
Column temperature: 30° C.
Autosampler temperature: 5° C.
Wavelength: 248 nm
Retention time: 20 min

For the analyses an HPLC Agilent model 1200 with diode array type detector, model G1315C was used.

Samples for analyses of the active ingredient content were obtained by dissolving in the solvent an amount of powder composition such as to obtain a concentration between 500 μg/ml and 600 μg/ml for tigecycline, as for reference solution.

Samples for the analysis of the active ingredient content were used for the analysis of impurities.

The reference solution was injected three consecutive times before the sample for determining the precision of the system, expressed as relative standard deviation percentage (RSD %) which must be lower than 2%.

Active ingredient content is obtained by the area ratio to the area of the tigecycline peak in the reference solution at known concentration. Product degradation is calculated as the ratio of the sum of the areas of the analytical peaks corresponding to the degradation products, corrected for each response factor, to the total area (active+impurities) in the sample. All analytical peaks whose area was greater than 0.1 percent of the total area are counted within the sum of degradation products.

3. Breathability Test with NGI (Next Generation Impactor)

The Next Generation Impactor (NGI) is an impactor for powders, described in pharmacopeia (EP; USP), used for the measurement of the aerodynamic diameter of powder particles dispersed in the air in the form of aerosol.

An inhalation formulation, delivered by a suitable inhaler and conveyed into the device by suction, deposits in various stages of the impactor, arranged in series, as a function of its aerodynamic characteristics, which depend on the sizes of particles, density and shape. At each stage of NGI corresponds a range of aerodynamic particle sizes of the powder deposited within it, determined through quantitative analysis by UV of the present active ingredient.

Through the quantitative determination of the active ingredient in each stage the aerodynamic size distribution of the powder is obtained and the mean aerodynamic diameter and the respirable fraction, defined by the European Pharmacopeia as the fraction having aerodynamic diameter less than 5.0 μm, can be calculated.

For the breathability test, powders of the formulations of examples were divided in capsules of HPMC size 3 and delivered through a powder inhaler RS01-model 7 single-dose, cod. 239700001AB (Aerolizer-Plastiape S.p.A.).

The device was assembled according to the user instructions and the indications of European Pharmacopeia.

From the analytical point of view, for the execution of the test, the delivery of a single powder capsule for each test of breathability is sufficient. Tests were performed at a flow rate of 60 Lpm for 4 seconds derived from a pressure drop in the system of 1.4 KPa.

To this flow rate for each stage of NGI correspond the following cut-offs of aerodynamic diameter.
stage 1: >8.06 μm
stage 2: between 8.06 μm and 4.46 μm
stage 3: between 4.46 μm and 2.82 μm
stage 4: between 2.82 μm and 1.66 μm
stage 5: between 1.66 μm and 0.94 μm
stage 6: between 0.94 μm and 0.55 μm
stage 7: between 0.55 μm and 0.34 μm
stage 8 (MOC): <0.34 μm

The respirable fraction (Fine Particle Fraction) is the drug amount, calculated relative to the delivered dose, characterized by particles having mean aerodynamic diameter lower than 5.0 μm and is calculated through a suitable validated software (CITDAS Copley).

The aerodynamic parameters of an inhalation formulation subjected to NGI analysis, are expressed in terms of:
Delivered Fraction (DF): i.e. the percentage of the active ingredient dose delivered outside the inhaler mouthpiece, relative to the loaded dose.
Fine Particle Dose (FPD): theoretically respirable dose of active substance, characterized by an aerodynamic diameter of less than 5.0 μm.
Fine Particle Fraction (FPF): theoretically respirable fraction (aerodynamic diameter less than 5.0 μm) of active substance expressed as a percentage of the amount delivered.
Mass Median Aerodynamic Diameter (MMAD): median aerodynamic diameter of particles delivered.
Geometric Standard Deviation (GSD): Geometric Standard Deviation relative to the median aerodynamic diameter.

The quantitative determination of active ingredient in each stage was performed through an UV spectrophotometry using the analytical method described below:
Solvent: 80/20 phosphate buffer pH 8/acetonitrile
Cuvettes for analysis: plastic, disposable, with optical path of 10 mm
Wavelength: 411 nm

For the analyses, an Agilent model Cary 3500 multicell spectrophotometer was used.

Samples for the analyses of the active ingredient content, deriving from the NGI test, were obtained by using solvent volumes such as to obtain a concentration range between 0.4 μg/ml and 60 μg/ml for tigecycline, with a reference solution having a tigecycline concentration of about 25 μg/ml.

The active ingredient content is obtained by ratio of the absorbance relative to the absorbance of the tigecycline peak in the reference solution at known concentration.

### Example 3

### Characterization of Powder: Determination of the Solid State Through X-Ray Diffractometry

### X-Ray Diffractometry Measurement

X-ray diffractometry measurements were performed for the determination of the solid state of the powder.

Crystals diffract X-rays in a manner characteristic of their structure. For this reason, the technique of X-ray diffractometry permits to determine the crystalline or amorphous solid state of the components of the sample.

The instrument used is the D2-Phaser from Bruker AXS with LYNXEYE sensor, DIFFRAC.MEASUREMENT CENTER.V7 measurement software.

Powder samples were arranged in a uniform layer on silicon sample holders with diameter of 20 mm and thickness of 0.5 mm.

The selected method of analysis uses the following instrument conformation:
Source: copper
Divergence Slit: 0.2 mm
Soller Slit: 4°
Knife: 1 mm

The scanning parameters were the following:
Angle range: 3-50° 2 Theta
Pitch length between measures: 0.02°
Dwell time at each angle: 1s
Detector aperture: 4 mm
Sample rotation: 15 rpm

FIG. 2 shows the diffractogram obtained with a powder comprising tigecycline and lactose in a 1:1 ratio (curve A) and with a powder comprising tigecycline, lactose and leucine in a ratio 1:1:1 (curve B).

As easily noted, curve A does not show any peak of a crystalline nature, resulting in the powder being substantially in amorphous form. Curve B shows two peaks attributable to leucine that reveals its alignment with a tendency to crystallization.

### Example 4

### Assessment of the Anti-Mycobacterial Activity

The test was conducted for assessing the cytotoxic activity and the anti-mycobacterial activity against non-tuberculous mycobacteria (NTM) (Mycobacterium abscessus) in the cell line of THP-1 macrophages of the formulation 2 of the example 1 constituted by tigecycline/lactose/L-leucine (30/60/10 w/w/w) relative to the pure active ingredient tigecycline.

### Complete DMEM Medium Used for Macrophage Composition

Modification of the Dulbecco Eagle medium (Cellgro 15-017-cv) supplemented with:
Heat-inactivated fetal calf serum (Atlas Biologicals, Fort Collins, CO, F-0500-A) (10%)
LCM (10%)

Medium conditioned by L929: L-929 (CCL-1) (ATCC) cells secrete the macrophage colony-stimulating factor (M-CSF), the cytokine that differentiates bone marrow macrophages/monocyte progenitors into a homogeneous population of mature macrophages. They are seeded at 4.7×10⁵ cells in 55 ml of DMEM+10% of fetal calf serum in a ball of 75 cm₂. Cells are left to grow for 3 days and then the supernatant is collected and filtered through a 0.45 μm filter, aliquoted and frozen at −20° C.

The cell-free filtrate is used in DMEM media.
L-glutamine (Sigma G-7513) (2 mM)
HEPES buffer (Sigma H-0887) (10 mM) Antibiotic/antimycotic (Sigma A-9909) (1×)
non-essential amino acids MEM (Sigma M-7145) (1×)
2-mercaptoethanol (Sigma M-6250) (50 nM)

### Preparation and Culture of the Macrophages Cell Line THP-1

THP-1 cells were expanded for 2 weeks. Subsequently, THP-1 cells were suspended in a complete DMEM medium (for macrophages at a concentration of 5×10⁵ cells/mL). Cells were seeded in 24-well tissue culture plates, 2 mL per well (1×106 per well).

The 24-well tissue culture plate permits to test in triplicate a range of 7 drug concentrations and non-treated controls. Cells were incubated at 37° C. with 5% CO₂ in a humidified chamber.

Complete DMEM media free of antibiotic/antimycotic were not changed during the 3-day test.

### Infection and Treatment of THP-1 Macrophages

On day 0, the media were removed from the cells and replaced with 0.2 mL of antibiotic/antimycotic-free DMEM containing Mycobacterium abscessus at a ratio of 10 bacteria per macrophage.

The tissue culture plates were placed inside closed Ziploc bags and transported to the incubator. Once inside the incubator, the bags were opened. The cells were incubated with the bacteria for 2 hours.

After the infection, the extracellular bacteria were removed by washing each well once with PBS. Then 2 mL of complete DMEM medium free of antibiotics/antimycotics and various drug concentrations were added.

For preparing drug concentrations, 2-times serial dilutions were executed by adding 10 ml of the previous suspension to 10 ml of complete medium plus serum in the next vial, resulting in a test range of 0.25, 1, 4 and 16x MIC.

Each drug concentration was tested in triplicate. Culture plates were incubated with antibiotics at 37° C.+5% CO₂ for 3 days.

After 3 days, cells were treated with gentamicin for 2 hours for killing the extra-cell bacteria and then washed 3 times with media. The plating of infected cell lysates and exposed to formulation 2 and to tigecycline raw material and the assessment of cell viability for THP-1 cells were performed after 4 hours, 1 day and 2 days.

The results are summarized in the following tables 6 and 7.

TABLE 6
Results with pure tigecycline

| Pure | | | |
|---|---|---|---|
| Tigecycline | Day 1 | Day 2 | Day 3 |
| μg/ml | CFU | CFU | CFU |
| 0 | 217 | 1233 | 13,333 |
| 1 | 287 | 2067 | 2667 |
| 2 | 263 | 203 | 1743 |
| 4 | 427 | 123 | 267 |
| 8 | 303 | 203 | 220 |
| 16 | 333 | 203 | 153 |
| 24 | 287 | 90 | 143 |

TABLE 7
Results with formulation 1

| Formulation 1 | Tigecycline | Day 1 | Day 2 | Day 3 |
|---|---|---|---|---|
| μg/ml | μg/ml | CFU | CFU | CFU |
| 0 | 0 | 217 | 1233 | 13,333 |
| 1 | 0.3 | 123 | 203 | 3300 |
| 2 | 0.6 | 170 | 163 | 2600 |
| 4 | 1.2 | 177 | 160 | 2000 |
| 8 | 2.4 | 163 | 183 | 517 |
| 16 | 4.8 | 193 | 147 | 497 |
| 24 | 7.2 | 163 | 183 | 160 |

The test showed a relevant perfusion of tigecycline within infected macrophages both from the pure active ingredient and the formulation 1.

The comparison between the efficiency of formulation 1 relative to the pure active ingredient highlighted a substantial equivalence of behavior between the two preparations taking into account that the quantity of tigecycline in the formulation 1 is equal to the 30% of the total.

In case of formulation 1 the maximum concentration of tigecycline used was equal to 7.2 μg/ml well below what was assessed with pure tigecycline.

Nevertheless, the reduction of CFU of Mycobacterium abscessus is evident even at low concentrations of tigecycline thus showing comparable dissolution rates between formulation 1 and active ingredient and above all the stability of tigecycline after the preparation by spray-drying.

The cytotoxicity test performed preliminarily with the formulation 1 has showed no toxicity. The macrophages exposed to the formulation 1 have maintained the 95% vitality after the exposure.

1: A pharmaceutical composition in the form of a dry powder for inhalation administration comprising a glycylcycline, in an amount of less than 50% by weight relative to the total weight of said composition, and lactose in an amount equal to or greater than the amount of said glycylcycline.

2: A pharmaceutical composition according to claim 1, wherein the weight ratio of the amount of glycylcycline to the amount of lactose is between 1:1 and 1:3.

3: A pharmaceutical composition according to claim 1, wherein said composition further comprises leucine.

4: A pharmaceutical composition according to claim 3, wherein said composition comprises said leucine in an amount of 5% to 30% by weight relative to the total weight of said composition.

5: A pharmaceutical composition according to claim 1, wherein said powder has an X₉₀ lower than 10.0 μm.

6: A pharmaceutical composition according to claim 1, wherein said powder has an MMAD lower than 5 μm.

7: A pharmaceutical composition according to claim 1, wherein said powder has a respirable fraction (FPF) greater than 50%.

8: A pharmaceutical composition according to claim 1, wherein said composition comprises said glycylcycline in an amount of 15% to 45%, by weight relative to the total weight of said composition.

9: A pharmaceutical composition according to claim 1, wherein said composition comprises said lactose in an amount of 30% to 85% by weight relative to the total weight of said composition.

10: A pharmaceutical composition according to claim 1, wherein said glycylcycline is present in the amorphous solid state in an amount of 90 to 100% relative to the total amount thereof in said composition.

11: A process for preparing a pharmaceutical composition in the form of a dry powder for inhalation administration, comprising a glycylcycline, wherein said process comprises:
(a) preparing a solution in an aqueous solvent comprising a glycylcycline, lactose, and optionally leucine,
(b) drying the solution of (a) to obtain a dry powder with X₉₀ lower than 10 μm, and
(c) collecting said dry powder,
characterized in that said solution further comprises an organic or inorganic acid in an amount necessary to give said solution a pH value between 6.5 and 7.5.

12: A process according to claim 11, wherein said (b) drying is carried out by means of a spray dryer.

13: A process according to claim 11, wherein said organic or inorganic acid is selected from the group consisting of formic acid, acetic acid, propionic acid, butyric acid, hydrochloric acid, hydrobromic acid, nitric acid and phosphoric acid.

14: A method for the treatment of a mycobacterial infection, comprising administering to a subject in need thereof an effective amount of a pharmaceutical composition in the form of a dry powder, wherein said composition comprises a glycylcycline, in an amount of less than 50% by weight relative to the total weight of said composition, lactose in an amount equal to or greater than the amount of said glycylcycline, and optionally leucine.

15: The method according to claim 14, wherein said mycobacterial infection is an infection of non-tuberculous mycobacteria (NTM).

16: A method according to claim 14, wherein said mycobacterial infection is an infection of a non-tuberculous mycobacteria (NTM) selected from the group consisting of slowly-growing NTM (SGM) and rapidly-growing NTM (RGM).

17: A method according to claim 14, wherein the weight ratio of the amount of glycylcycline to the amount of lactose is between 1:1 and 1:3.

18: A method according to claim 14, wherein said composition further comprises leucine.

19: A method according to claim 18, wherein said composition comprises said leucine in an amount of 5% to 30% by weight relative to the total weight of said composition.

20: A method according to claim 14, wherein said powder has an X₉₀ lower than 10.0 μm.
